(19) United States
(12) Patent Application Publication
(10) Pub. No.: US 2024/0335383 A1
(43) Pub. Date: Oct. 10, 2024

(54) NANOPARTICULATE FORMULATION

(71) Applicant: NewImmune II, LLC, Wilmington, DE (US)

(72) Inventors: Brian HORSBURGH, New Canaan, CT (US); Mark David MOODY, Concord, MA (US)

(73) Assignee: NewImmune II, LLC, Wilmington, DE (US)

(21) Appl. No.: 18/700,066

(22) PCT Filed: Oct. 11, 2022
(86) PCT No.: PCT/GB2022/052570

§ 371 Date: Apr. 10, 2024

Related U.S. Application Data
(60) Provisional application No. 63/321,886, filed on Mar. 21, 2022.
(60) Provisional application No. 63/254,253, filed on Oct. 11, 2021.

(30) Foreign Application Priority Data
Feb. 2, 2022 (GB) 2201351.0

(51) Int. Cl. A61K 9/127; A61K 31/4745; A61K 38/20; A61K 47/10; A61K 47/24; A61K 47/28; A61K 47/69; A61P 35/00

(52) U.S. Cl. CPC A61K 9/1271; A61K 31/4745; A61K 38/2013; A61K 47/10; A61K 47/24; A61K 47/28; A61K 47/6951; A61P 35/00

(57) ABSTRACT

The present disclosure relates to nanoparticles and nanoparticulate compositions; methods of preparing such nanoparticles and nanoparticulate compositions; and associated methods of medical treatment and uses of such nanoparticles and nanoparticulate compositions for medical treatment, including the use of such nanoparticles for the manufacture of medicaments for medical treatment.

### FIELD OF THE DISCLOSURE

The present disclosure relates to nanoparticles and nanoparticulate compositions; methods of preparing such nanoparticles and nanoparticulate compositions; and associated methods of medical treatment and uses of such nanoparticles and nanoparticulate compositions for medical treatment, including the use of such nanoparticles for the manufacture of medicaments for medical treatment. Nanoparticles and nanoparticulate compositions disclosed herein may be effective for stimulating or enhancing a therapeutic or protective immune response in a patient, and may be used for the treatment of cancer, including metastatic cancer. Nanoparticles and nanoparticulate compositions disclosed herein may be used for the treatment of vulnerable patient groups, including immunosuppressed patients, such as patients who are immunosuppressed by the action of immunosuppressive medication administered to the patient following an organ or multi-organ transplant.

### BACKGROUND

Imiquimod (R-837)—also known as 1-(2-methylpropyl)imidazo[4,5-c]quinolin-4-amine (CAS Number: 99011-02-6) and S-26308—is a small molecule imidazoquinoline drug having the structural formula:

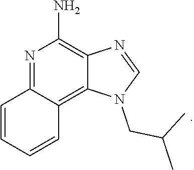 [SMILES: CC(C)Cn1cnc2c(N)nc3ccccc3c21]

Imiquimod stimulates the innate and adaptive immune system by activating toll-like receptors 7 and/or 8 (TLR7/8). When administered in vivo, imiquimod is capable of triggering TLR7-mediated production of IFNα, TNFα, and IL-12; inducing the recruitment of antigen-presenting plasmacytoid dendritic cells; and promoting CTL responses. Studies have shown that imiquimod is capable of activating primary macrophages to an M1 pro-inflammatory immunophenotype, which is associated with improved clinical outcomes in cancer patients.

Various active structural analogues of imiquimod (R-837) have been synthesized and characterised. These include the imidazoquinolines resiquimod (R-848), gardiquimod, CL097, S28690, 852-A, and 854A, the thiazoloquinolone CL075, and others, including those in Table 1 below:

TABLE 1

| Structural | |
|---|---|
| analogue | Molecular structure |
| Resiquimod | 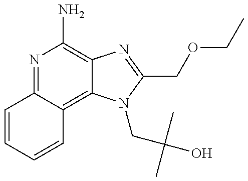 [SMILES: CCOCc1nc2c(N)nc3ccccc3c2n1CC(C)(C)O] |
| Gardiquimod | 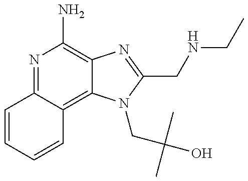 [SMILES: CCNCc1nc2c(N)nc3ccccc3c2n1CC(C)(C)O] |
| CL097 | 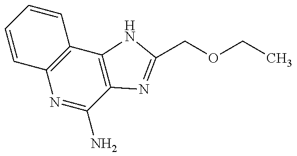 [SMILES: CCOCc1nc2c(N)nc3ccccc3c2[nH]1] |
| 852-A (PF- 4878691) | 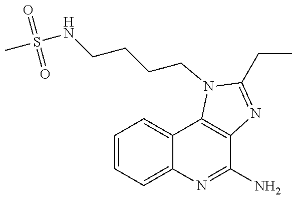 [SMILES: CCc1nc2c(N)nc3ccccc3c2n1CCCCNS(C)(=O)=O] |
| 854A (3M-011) | 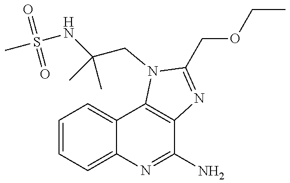 [SMILES: CCOCc1nc2c(N)nc3ccccc3c2n1CC(C)(C)NS(C)(=O)=O] |
| CL075 (3M-002) | 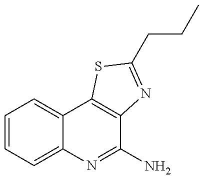 [SMILES: CCCc1nc2c(N)nc3ccccc3c2s1] |

Another exemplary structural analogue of imiquimod (R-837) is 528690, a small molecule TLR7 agonist described in Hicks et al, Blood (2004) 104 (11):3481.

These structural analogues of imiquimod (R-837) are active TLR7/8 ligands which have similar properties and activity to imiquimod (R-837), optionally including pH-dependent solubility. In this disclosure, the term “imiquimod” henceforth denotes imiquimod (R-837); but also embraces and refers to structural analogues of imiquimod (R-837) which are TLR7/8 agonists or TLR7/8 ligands, including but not limited to those listed above. Suitably, structural analogues of imiquimod (R-837) may display pH-dependent solubility, with increased solubility at lower pH. The term “imiquimod” as used herein accordingly includes imidazoquinolines which are active TLR7/8 ligands and which have the basic molecular structure:

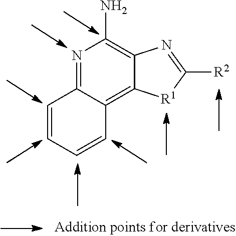

where R¹ is typically N and R² is typically H or C and where the imidazoquinoline is optionally substituted at one or more of the indicated addition points with one or more substituents, which substituents may be independently selected from branched, linear or cyclic alkyl, alkenyl, alcohol, alkylamine, alkoxy or alkoxyalkyl groups, in particular, C₁₋₁₀ alkyl, alkenyl, alcohol, alkylamine, alkoxy or alkoxyalkyl groups, or hydroxyl groups, or amine groups, or N—(C₁₋₁₀ alkyl)methanesulphonamide groups. The term “imiquimod” as used herein also includes close structural derivatives of these imidazoquinolines, including thiazoloquionoline derivatives, which are active TLR7/8 ligands.

Imiquimod has been shown to be effective against skin cancers and precancerous lesions, especially basal cell cancers and actinic keratosis. It is approved by the FDA as an active ingredient in two topical cream formulations, Aldara® and Zyclara®. Aldara® cream contains 5% imiquimod (R-837), and is approved for topical treatment of rough, raised areas of heavily sun-exposed skin (actinic keratoses), skin cancer (basal cell carcinoma), and external genital and perianal warts/condyloma acuminata. Zyclara® cream contains 3.75% imiquimod, and is approved for topical treatment of actinic keratoses and external genital and perianal warts/condyloma acuminata.

IL-2, or interleukin-2, is also known for use in cancer immunotherapy. IL-2 is a cytokine with pleiotropic effects on the immune system. It can induce and potentiate natural killer (NK) cells and can act as a growth factor for T cells, stimulating and enhancing an anti-cancer immune response. IL-2 has been approved by the FDA in recombinant form, known as aldesleukin and sold as Proleukin®, for treatment of metastatic renal cell carcinoma and metastatic melanoma by systemic injection or infusion. It is also available for intralesional use. Since 2016, intralesional IL-2 has been supported by NCCN clinical guidance practice guidelines as an effective treatment for in-transit non-resectable melanoma.

Studies have shown that patients with in-transit metastases of malignant melanoma can be effectively treated using a combination of topical imiquimod cream, applied to accessible cutaneous lesions, with intralesionally injected IL-2. Green et al (British J of Dermatol 2007 156:337-345) reports that the combination of topical imiquimod cream and intralesional IL-2 is relatively simple and is well tolerated, and may provide a useful additional option for the palliative management of in-transit metastatic melanoma. Similar results have been reported by Shi et al, J Amer. Acad. Dermatol. 73(4) 2015: 645-654 and by Leventhal et al, JAAD Case Reports 2016; 2:114-6, suggesting that combination therapy using topical imiquimod and tretinoin cream in conjunction with intralesional IL-2 may increase the anti-tumor efficacy of the IL-2.

A recent case study has reported the combination of intralesional IL-2 and topical imiquimod to be safe and effective for clearing multifocal, high grade squamous cell cancer in a combined liver and kidney transplant patient, with no evidence of immune-mediated organ rejection (Vidovic et al, Frontiers in Immunology. 2021 12:1957-1967). This report is particularly interesting, as there is a wider clinical need for safe and effective cancer therapies in organ transplant patients. Patients who undergo organ transplants are at increased risk of developing cancer—often skin cancer—that can be both aggressive and deadly, showing a higher than average propensity for rapid development, recurrence and metastasis. As a result, the median survival time following a cancer diagnosis in a transplant patient is currently only 2.7 years, compared to a median of 8.3 years post-diagnosis survival in their immunocompetent counterparts. This is primarily attributable to immunosuppression caused by anti-rejection medications, which blunt the immune's system's ability to recognize and eliminate cancer, allowing cancer in these patients to undergo immune escape and develop and spread more aggressively. In addition to their heightened vulnerability to cancer, transplant patients face a paucity of cancer treatment options, as chemotherapy can be damaging to the transplanted organ. The report by Vidovic et al is therefore clinically significant, in identifying that combination therapy with IL-2 and imiquimod may be both safe and effective for treatment of multiple cancerous lesions in an immunocompromised multi-organ transplant patient.

The present inventors have recognised that imiquimod and a protein cytokine such as IL-2 is a promising drug combination for more general use in immunotherapy; particularly cancer immunotherapy; including in immunosuppressed patient groups, including organ transplant patients. The available clinical formulations and characteristics of these APIs however limit the current therapeutic utility of this drug combination. Imiquimod (R-837) is currently approved and available only as a topical cream, which limits its therapeutic use to cutaneous applications. IL-2 is available in injectable form for systemic intravenous use or localised injection, and is therefore separately administered; this is inconvenient and undesirable for the patient and the healthcare practitioner. The short in vivo half-lives of imiquimod and IL-2 also pose a challenge. IL-2 has a very short in vivo half-life of <10 minutes, against an in vivo half-life of approximately 3 hours for imiquimod. This suggests a very limited window of time for simultaneous/synergistic therapeutic action following co-administration of these active agents.

Appreciating these issues, the present inventors have recognised that it would be desirable to provide a combined formulation of imiquimod and a protein cytokine such as IL-2, which is suitable for parenteral administration and is capable of achieving controlled and, optionally, sustained delivery of imiquimod and the protein cytokine, such as IL-2, for the purposes of combination immunotherapy; particularly for the purposes of treating cancer; and/or for the treatment of immunosuppressed patients, including organ transplant patients.

This has not previously been recognised as a desirable objective; and it has not proved a straightforward endeavour. Imiquimod presents a challenge for parenteral formulation and administration, primarily owing to its very low solubility in aqueous solution at physiological pH. As shown in FIG. 1—a graph illustrating the aqueous solubility of imiquimod as a function of pH using Britton-Robinson buffer—imiquimod shows maximum solubility in aqueous solution at pH 2 or below. The aqueous solubility of imiquimod decreases sharply with increasing pH above pH 2, and it is only sparingly soluble above pH 6. For this reason, efforts to develop aqueous or aqueous-based formulations of imiquimod have typically involved the use of acidic solvents below pH 4. Hayashi et al (Int. J. Urol. 2010 May; 17(5):483-90) describes a formulation of imiquimod for intrathecal administration, which uses 0.1 M lactic acid, poloxamer, and HP-β-CD at low acidic pH. Guedes et al, (J. Braz. Chem. Soc., Vol. 31, No. 8, 1732-1745, 2020) similarly describes the solubilisation of imiquimod in β-cyclodextrin in the presence of citric acid at pH 3. Ramineni et al (J. Pharm. Sci. 2013 February; 102(2):593-603) developed mucoadhesive films containing imiquimod and HP-β-CD, using a mixture of acetate buffer and methanol at pH 4. Fox et al (Journal of Nanobiotechnology 2014, 12:17) meanwhile discloses an anionic liposomal formulation comprising imiquimod solubilised in a lactic acid core at pH 2.5-3.5.

Whilst imiquimod can be formulated in aqueous-based formulations at low pH as described in the above and similar references, this approach does not readily allow for co-formulation of the imiquimod with a protein cytokine such as IL-2, which denatures at low pH. Low pH formulations are also unsuitable for parenteral administration.

Against this backdrop, the present inventors have successfully developed and describe herein a nanoparticle and nanoparticulate formulation containing imiquimod, which nanoparticle or nanoparticulate formulation is suitable for parenteral administration and may allow for sustained release of the imiquimod from the nanoparticle or nanoparticulate formulation.

The present disclosure further provides a nanoparticle and nanoparticulate formulation comprising imiquimod and a protein cytokine such as IL-2, which nanoparticle or nanoparticulate formulation is suitable for parenteral administration, and may allow for sustained release of both APIs (imiquimod and protein cytokine) from the nanoparticle or nanoparticulate formulation. The disclosed nanoparticles and nanoparticulate formulations may be effectively used for the purposes of immunotherapy, particularly cancer immunotherapy, in patients who will benefit from combined delivery of imiquimod and a protein cytokine such as IL-2.

### SUMMARY OF THE DISCLOSURE

According to one aspect, the present disclosure provides a nanoparticle for co-delivery and, optionally, sustained release of imiquimod and a protein cytokine such as IL-2, comprising an outer lipid shell and an inner aqueous core encapsulated within the outer lipid shell; wherein the inner aqueous core comprises imiquimod; and wherein the nanoparticle further comprises the protein cytokine, such as IL-2, releasably attached to, associated with and/or encapsulated within the lipid shell.

According to another aspect, the present disclosure provides a nanoparticle for delivery and, optionally, sustained release of imiquimod, comprising an outer lipid shell and an inner aqueous core encapsulated within the outer lipid shell; wherein the inner aqueous core comprises imiquimod and a host molecule that is capable of reversibly forming a complex with imiquimod; and wherein the inner aqueous core has a pH of about 6.5 or above.

In a further aspect, the present disclosure provides a nanoparticulate composition comprising a plurality of nanoparticles in accordance with the present disclosure. The nanoparticulate composition may be or may comprise an aqueous solution, aqueous dispersion or aqueous suspension of nanoparticles as disclosed.

In a further aspect, the present disclosure provides a nanoparticulate composition as disclosed, for use in stimulating or enhancing a therapeutic or protective immune response. Specifically, the disclosure provides a nanoparticulate composition as disclosed, for use in the treatment of cancer. The present disclosure further provides a method of stimulating or enhancing a therapeutic or protective immune response, comprising the step of administering a nanoparticulate composition as disclosed herein to a patient in need thereof. The disclosure provides a method for treating cancer, comprising the step of administering a nanoparticulate composition as disclosed herein to a patient in need thereof.

The present disclosure further provides a method for manufacturing a nanoparticle as disclosed, comprising the sequential steps of:
(a) solubilising imiquimod in aqueous solution at a pH buffered to about pH 6 or below; followed by
(b) combining the resulting aqueous solution with lipids to form lipid shell nanoparticles encapsulating imiquimod; followed by
(c) increasing the buffered pH of the formulation to about pH 6.5 or above.

Step (c) may optionally be followed by
(d) adding a protein cytokine such as IL-2 to the formulation of (c) to load the nanoparticles with the protein cytokine.

The disclosed nanoparticulate compositions may be administered to a patient parenterally, optionally by intralesional or intravenous injection; and may be used for the treatment of a wide range of conditions, including cancers, which are responsive to immunotherapy. The compositions may be used, in particular, for intralesional injection into clinically palpable cutaneous and/or subcutaneous metastatic lesions in patients with advanced malignant melanoma who have exhausted all standard therapies. The compositions may be used for the treatment of immunosuppressed patients; particularly for treatment of patients who are immunosuppressed by action of anti-rejection medication administered following an organ transplant.

Nanoparticles according to the present disclosure have been designed to facilitate coordinated, controlled, sustained and optionally localised delivery of imiquimod and, optionally, a protein cytokine such as IL-2, to a patient in need of immunotherapy. This may improve the pharmacokinetics of both APIs, while providing therapeutic benefit at doses which may in some instances be substantially below the approved therapeutic doses of imiquimod and IL-2, thereby improving patient safety.

The disclosed nanoparticulate compositions comprise the small molecule imiquimod, optionally together with a protein cytokine such as IL-2, encapsulated in liposomes. These active molecules are contained in approved and marketed drugs (Aldara® and Proleukin®, respectively) and are well characterised and well understood by medical oncologists. There have been several clinical trials evaluating the drug combination. In these trials, IL-2 was injected into tumors, and imiquimod was separately applied as a topical cream to the skin; the response rates were between 41 and 100% and the drugs were generally well tolerated. The mechanism of therapeutic action for the drug combination is thought to be accomplished by the immune-stimulatory effects of IL-2, and the associated effects of imiquimod activating the innate immune system.

Whilst the use of protein cytokines such as IL-2 in conjunction with imiquimod for cancer immunotherapy has been previously described, a combined parenteral formulation which allows for simultaneous administration and controlled delivery of both drugs has not previously been available in the art. Further, a nanoparticulate formulation suitable for parenteral administration of imiquimod as disclosed, and a method for producing such a nanoparticulate formulation, has not previously been available in the art.

### BRIEF DESCRIPTION OF THE FIGURES

FIG. 1 shows a graph illustrating the effect of pH on the solubility of imiquimod in aqueous solution.

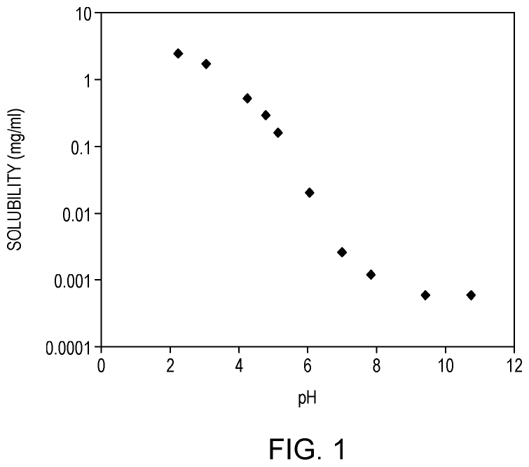

FIG. 2 shows the structure of an exemplary nanoparticle according to the present disclosure.

FIG. 3 shows a Cryo TEM image of nanoparticles according to the present disclosure.

FIG. 4 shows a flow chart illustrating the steps of an exemplary method for production of nanoparticles according to the present disclosure.

FIG. 5 shows the results of a release assay illustrating the release profile of imiquimod from nanoparticles according to the present disclosure.

### DETAILED DESCRIPTION

In this disclosure:
“imiquimod” denotes imiquimod (R-837); but also embraces and refers to structural analogues of imiquimod (R-837) as defined hereinabove, which are TLR7/8 agonists or TLR7/8 ligands and which may display pH-dependent solubility, including but not limited to imidazoquinolines and thiazoloquinolones as shown in Table 1;
“hydrogel” denotes a matrix of water-swellable hydrophilic polymers, optionally cross-linked; and
“immune response” denotes any protective and/or defensive response of the immune system.

The present disclosure provides a nanoparticle for co-delivery and, optionally, sustained release of imiquimod and a protein cytokine such as IL-2, comprising an outer lipid shell and an inner aqueous core encapsulated within the outer lipid shell; wherein the inner aqueous core comprises imiquimod; and wherein the nanoparticle further comprises a protein cytokine such as IL-2 releasably attached to, associated with and/or encapsulated within the lipid shell. The present disclosure further provides a nanoparticle for delivery and, optionally, sustained release of imiquimod, comprising an outer lipid shell and an inner aqueous core encapsulated within the outer lipid shell; wherein the inner aqueous core comprises imiquimod and a host molecule that is capable of reversibly forming a complex with imiquimod; and wherein the inner aqueous core has a pH of at least about 6.5. The inner aqueous core of the nanoparticles according to this disclosure may optionally have a pH of 7 or above, or a pH of 7.5 or above; and optionally may have a pH no more than about pH 9, or a pH no more than about pH 8.5; suitably a pH between about 6.5-9 or between about 6.5-8.5 or between about pH 6.5-8 or between about pH 7-9 or between about pH 7-8.5 or between about pH 7-8, or between about pH 7.5-9.

The outer lipid shell of the nanoparticles of this disclosure comprises one or more lipid layers or bilayers, which enclose a central core. The lipids forming the shell may be neutral, zwitterionic, anionic or cationic lipids at physiologic pH. The lipids within and/or between each lipid layer or bilayer, may, optionally, be cross-linked. The outer lipid shell may accordingly be composed of one or more concentric lipid layers, optionally crosslinked, wherein the lipids can be neutral, anionic or cationic lipids at physiologic pH. The composition of the lipid shell and the extent of cross-linking within or between the lipid layers can be varied in order to modify and optimise the release profiles of the active agents (imiquimod and/or protein cytokine).

In some favoured embodiments, the outer lipid shell of a nanoparticle according to this disclosure may comprise lipids selected from the group consisting of cholesterol, phospholipids, lysolipids, lysophospholipids, and sphingolipids, and derivatives thereof. Suitable lipids include, but are not limited to, phosphatidylcholine (PC) (such as egg PC, soy PC), including 1,2-diacyl-glycero-3-phosphocholines; phosphatidylserine (PS); phosphatidylglycerol; phosphatidylinositol (PI); glycolipids; sphingophospholipids, such as sphingomyelin; sphingoglycolipids (also known as 1-ceramidyl glucosides), such as ceramide galactopyranoside, gangliosides and cerebrosides; fatty acids; sterols containing a carboxylic acid group such as cholesterol or derivatives thereof; and 1,2-diacyl-sn-glycero-3-phosphoethanolamines, including 1,2-dioleoyl-sn-glycero-3-phosphoethanolamine or 1,2-dioleolylglyceryl phosphatidylethanolamine (DOPE), 1,2-dihexadecylphosphoethanolamine (DUPE), 1,2-distearoylphosphatidylcholine (DSPC), 1,2-dipalmitoylphosphatidylcholine (DPPC), and 1,2-dimyristoylphosphatidylcholine (DMPC). Suitable lipids also include natural lipids, such as tissue derived L-α-phosphatidyl: egg yolk, heart, brain, liver, soybean) and/or synthetic (e.g., saturated and unsaturated 1,2-diacyl-sn-glycero-3-phosphocholines, l-acyl-2-acyl-sn-glycero-3-phosphocholines, 1,2-diheptanoyl-SN-glycero-3-phosphocholine) derivatives of these lipids.

The outer lipid shell may also or alternatively comprise cationic lipids, including but not limited to N-[1-(2,3-dioleoyloxy)propyl]-N,N,N-trimethyl ammonium salts, also referred to as TAP lipids, for example as a methylsulfate salt. Suitable TAP lipids include, but are not limited to, DOTAP (dioleoyl-), DMTAP (dimyristoyl-), DPTAP (dipalmitoyl-), and DSTAP (distearoyl-). Other suitable cationic lipids include dimethyldioctadecyl ammonium bromide (DDAB), 1,2-diacyloxy-3-trimethylammonium propanes, N″ [1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dmiethylammonium propanes, dioctadecylamidoglycylspermine (DOGS), 3-[N—(N′ 5N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyltrimethylammonium bromide (CTAB), diC₁₄-amidine, N-tert-butyl-N′-tetradecyl-3-tetradecylamino-propionamidine, N-(alpha-trimethylammonioacetyl)didodecyl-D-glutamate chloride (TMAG), ditetradecanoyl-N-(trimethylarnmonio-acetyl)diethanolamine chloride, 1,3-dioleoyloxy-2-(6-carboxy-spermyl)-propylamide (DOSPER), and N,N,N′N′-tetramethyl-, N′-bis(2-hydroxylethyl)-2,3-dioleoyloxy-1,4-butanediammonium iodide, 1-[2-(acyloxy)ethyl]2-alkyl(alkenyl)-3-(2-hydroxyethyl)-imidazolinium chloride derivatives, such as 1-[2-(9(Z)-octadecenoyloxy)ethyl]-2-(8(Z)-heptadecenyl-3-(2-hydroxyethyl)imidazolinium chloride (DOTIM) and 1-[2-(hexadecanoyloxy)ethyl]-2-pentadecyl-3-(2-hydroxyethyl)imidazolinium chloride (DPTIM), and 2,3-dialkyloxypropyl quaternary ammonium derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

In some embodiments, the outer lipid shell of a nanoparticle according to this disclosure may comprise a PEGylated derivative of a neutral, cationic, anionic or zwitterionic lipid, such as mPEG-DSPE, including DSPE PEG (2000MW) and DSPE PEG (5000 MW). The surface display of PEG, or other suitable hydrophilic polyalkylene oxides, on the outer shell of the nanoparticle may serve to reduce uptake of the nanoparticle by the reticuloendothelial system (“RES”) when the nanoparticle is present in vivo; thereby prolonging in vivo residence and systemic circulation time and/or allowing the nanoparticle to provide a sustained and prolonged immunostimulatory effect. Further examples of suitable PEGylated lipids include dipalmitoyl-glycero-succinate polyethylene glycol (DPGS-PEG), stearyl-polyethylene glycol and cholesteryl-polyethylene glycol.

In some embodiments, the outer lipid shell may comprise a mixture of phospholipids and cholesterol, such as a mixture of N-(carbonyl-) methoxypolyethylene glycol 2000)-1,2-distearoyl-sn-glycero 3-phosphoethanolamine sodium salt (mPEG-DSPE), phosphatidyl choline such as fully hydrogenated soy phosphatidylcholine (HSPC), and cholesterol. These lipids are well known and well characterised, being used in approved commercial products such as Doxil®. Alternative suitable phospholipids, known to the skilled person, may be used in place of the DSPE-PEG and/or HSPC. The lipids may be mixed and used in any desired molar ratio. For example, the molar ratio of the phospholipids to the cholesterol may range from about 1:1 to about 6:1, more preferably from about 1:1 to about 3:1, most preferably about 2:1. Where the phospholipids include DSPE-PEG and HSPC, these components may be present in a molar ratio DSPE-PEG:HSPC of about 1:1-1:200 or 1:10-1:200; suitably from 1:1-1:100 or from 1:1-1:50 or from 1:1-1:30 or from 1: 10-1:30; advantageously from 1:15-1:25. In some embodiments, the molar ratio of the HSPC:DSPE-PEG:cholesterol may be about 2:0.1:1 or about 2:0.01:1 or about 2:0.2:1.

The outer lipid shell of a nanoparticle according to this disclosure encloses an inner aqueous core. In some embodiments, the inner aqueous core comprises one or more hydrogel polymers which may serve to stabilise and/or control the release of the active agents (imiquimod and/or protein cytokine) which may be comprised in or contained within the nanoparticle. The hydrogel polymers may be covalently and/or non-covalently cross-linked, or may be capable of being covalently and/or non-covalently cross-linked, or may have no cross-links. The hydrogel polymers may, for example, be or include poly(lactic acid), poly(glycolic acid), poly(lactic acid-co-glycolic acids), polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); poly(glycolide-co-caprolactones); polycarbonates; polyamides, polypeptides, and poly(amino acids); polyesteramides; other biocompatible polyesters; poly(dioxanones); poly(alkylene alkylates); hydrophilic polyethers; polyurethanes; polyetheresters; polyacetals; polycyanoacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), polyvinyl alcohols, polyvinylpyrrolidone; poly(alkylene oxides); celluloses, polyacrylic acids, albumin, collagen, gelatin, prolamines, and/or polysaccharides. In particular, the hydrogel polymers may include copolymers, including block copolymers, or blends of any of the aforementioned hydrogel polymers. In some favoured embodiments according to the present disclosure, the inner aqueous core of the nanoparticle comprises a polyethylene glycol polymer, such as polyethylene glycol 4000. PEG 4000 is widely used in pharmaceutical formulations, including parenteral formulations such as INVEGA SUSTENNA®. The inner aqueous core may additionally or alternatively comprise a block copolymer containing one or more poly(alkylene oxide) segments, such as polyethylene glycol, and one or more aliphatic polyester segments, such as polylactic acid.

The inner aqueous core of the nanoparticles of this disclosure comprises imiquimod. As mentioned above, the term “imiquimod” as used herein embraces the imidazoquinoline known as 1-(2-methylpropyl)imidazo[4,5-c]quinolin-4-amine (CAS Number: 99011-02-6), R-837, and 5-26308 and shown below:

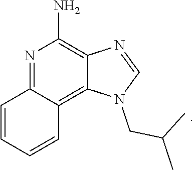 [SMILES: CC(C)Cn1cnc2c(N)nc3ccccc3c21]

The term further embraces structural analogues of imiquimod (R-837) as defined herein, which are active TLR7/8 ligands, including but not limited to resiquimod, gardiquimod, CL097, 528690, 852-A, 854A, CL075 and others known in the art and as defined above. In some preferred embodiments and aspects of this disclosure, the imiquimod is imiquimod (R-837).

Imiquimod as defined herein is a small synthetic guanosine analogue which is recognised for its immune-stimulating capabilities, and in particular is known to be effective in activating TLR7 and/or TLR8.

Imiquimod is approved for therapeutic administration as a cutaneous cream and is commercially available as a drug substance manufactured and tested in accordance with current Good Manufacturing Practices (cGMPs) under an active Drug Master File. Alternatively, imiquimod may be readily synthesised as a small molecule based on available raw materials and using methods well known in the art.

Suitably, the imiquimod may be dispersed, dissolved or suspended in the inner aqueous core of the nanoparticle, and/or may be present in the form of a precipitate. In some embodiments and aspects of the present disclosure, the inner aqueous core further comprises a host molecule that is capable of reversibly forming a complex, such as an inclusion complex, with imiquimod. In these embodiments, the aqueous core of the nanoparticle may comprise imiquimod complexed with a host molecule, and/or may comprise uncomplexed imiquimod and/or host molecule. An inclusion complex can be formed where an imiquimod molecule, or part of an imiquimod molecule, inserts into a cavity of a host molecule or group of host molecules. The host molecule may assist in solubilising the imiquimod in the aqueous core of the nanoparticle, and/or with controlling the release of the imiquimod from the nanoparticle. The host molecule may, for example, comprise a cyclodextrin; preferably a cyclodextrin selected from α-cyclodextrin; β-cyclodextrin; γ-cyclodextrin; methyl α-cyclodextrin; methyl β-cyclodextrin; methyl γ-cyclodextrin; ethyl β-cyclodextrin; butyl α-cyclodextrin; butyl β-cyclodextrin; butyl γ-cyclodextrin; pentyl γ-cyclodextrin; hydroxyethyl β-cyclodextrin; hydroxyethyl γ-cyclodextrin; 2-hydroxypropyl α-cyclodextrin; 2-hydroxypropyl β-cyclodextrin; 2-hydroxypropyl γ-cyclodextrin; 2-hydroxybutyl 3-cyclodextrin; acetyl α-cyclodextrin; acetyl β-cyclodextrin; acetyl γ-cyclodextrin; propionyl β-cyclodextrin; butyryl β-cyclodextrin; succinyl α-cyclodextrin; succinyl β-cyclodextrin; succinyl γ-cyclodextrin; benzoyl β-cyclodextrin; palmityl β-cyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; 3-cyclodextrin carboxymethylether; γ-cyclodextrin carboxymethylether; carboxymethylethyl β-cyclodextrin; phosphate ester α-cyclodextrin; phosphate ester β-cyclodextrin; phosphate ester γ-cyclodextrin; 3-trimethylammonium-2-hydroxypropyl β-cyclodextrin; sulfobutyl ether β-cyclodextrin; carboxymethyl α-cyclodextrin; carboxymethyl β-cyclodextrin; carboxymethyl γ-cyclodextrin, and combinations thereof. However, many other host molecules are known in the art and may be used in accordance with this disclosure, such as polysaccharides, cryptands, cryptophanes, cavitands, crown ethers, dendrimers, ion-exchange resins, calixarenes, valinomycins, nigericins, catenanes, polycatenanes, carcerands, cucurbiturils, and spherands, and others familiar to the skilled person.

In certain favoured embodiments, the host molecule is or includes 2-hydroxypropyl-β-cyclodextrin. 2-hydroxypropyl-β-cyclodextrin (HP-β-CD) (CAS number 128446-35-5) is a partially substituted poly(hydroxpropyl) ether of beta cyclodextrin (molar substitution 0.59-0.73 per anhydro glucose unit). It is capable of reversibly complexing with imiquimod, such as to improve the solubilisation of imiquimod in the aqueous inner core of the nanoparticle, whilst allowing for release of imiquimod from the nanoparticle under suitable conditions. HP-β-CD is currently used in a number of marketed products, including Mitozytrex™, a formulation of HP-β-CD and mitomycin approved for treatment of adenocarcinoma of the stomach or pancreas in the United States.

In some embodiments of this disclosure, the nanoparticle comprises a protein cytokine such as IL-2 releasably attached to, associated with and/or encapsulated within the lipid shell. IL-2, or interleukin-2 or aldesleukin, is a well-known and well-characterised protein cytokine which is readily available in recombinant form, including from Clinigen Inc under the brand name Proleukin®. Preferably the TL-2 is human IL-2. The IL-2 may be recombinant human IL-2, including biosimilar or biobetter human IL-2.

The protein cytokine may be encapsulated within the lipid shell, dispersed within the aqueous core; or may be releasably attached to or associated with the lipid shell. In some embodiments, the protein cytokine may be non-covalently attached to the lipid shell. Non-covalent attachment of IL-2 to a lipid bilayer may, for example, be effected by interaction of a tryptophan residue in the IL-2 with the lipid bilayer; as described in Koppenhagen et al, J. Pharm. Sci. 1998 June; 87(6):707-14. Here, recombinant IL-2 was shown to adsorb to liposomes through interaction of a single tryptophan with the lipid bilayer. In some other embodiments, the protein cytokine may be covalently attached to the lipid shell by way of a cleavable linking group, which linking group can be cleaved under suitable conditions, such as at a certain ambient pH or in the presence of certain cleaving agent(s), such as to release the protein cytokine.

In some embodiments, the nanoparticle may be decorated with one or more targeting agents displayed on the outer surface of the lipid shell. Targeting agents include proteins, peptides, lipids, nucleic acids, saccharides, or polysaccharides that bind to a target therapeutic substrate, such as a specific organ, tissue, cell, or extracellular matrix, or a specific type of tumor or infected cell. The display of an appropriately selected targeting agent on the surface of the nanoparticle can therefore assist in directing the nanoparticle towards its therapeutic target substrate in vivo. For example, where the therapeutic target substrate is a tumor, the targeting agent may be a polypeptide such as an antibody or antibody fragment which binds specifically to a tumor antigen or tumor marker. Suitable targeting molecules that can be used to direct nanoparticles to cells and tissues of interest, as well as methods of conjugating target molecules to nanoparticles, are known in the art. Nanoparticles according to the present disclosure may suitably be decorated with a targeting agent selected antibodies, aptamers, nanobodies, transferrins, CD13, RGD peptides, NGR peptides, folic acid and conjugates; particularly those listed in Table 2 below:

TABLE 2

| Class | Ligand | Targets |
|---|---|---|
| Antibodies | Rituxan | CD20 |
| | Trastuzumab, Herceptin | HER2 |
| | PD-L1, Keytruda, Opdivo | CD19 |
| | Ipilimumab | PD-1 |
| | | CD47 |
| | | CTLA-4 |
| | | LAG3 |
| Peptides | RGD peptide | αᵥβ₃ integrins |
| | NGR peptide | Aminopeptidase N |
| | Uroguanylin | Guanylate cyclase-C (GC-C) |
| | | receptors |
| Proteins | Transferrin LHRH | Transferrin receptor |
| | | LHRH receptor |
| Aptamers | Pegaptanib | VEGF receptor |
| | | PD-1 |
| | | CTLA4 |
| Small | Folate Galactose | Folate receptor |
| molecules | | Asialoglyco-protein receptor |
| | | VEGF receptor |

The nanoparticle may have a diameter, measured by the standard, art-recognised technique of Dynamic Light Scattering (DLS), of no more than about 300 nm. In some embodiments, the diameter of the nanoparticle (measured by DLS) may be no more than about 200 nm, or no more than about 150 nm, or no more than about 130 nm, or no more than about 120 nm. The diameter of the nanoparticle (measured by DLS) may be at least i5 nm or at least 20 nm or at least 30 nm or at least 50 nm. Suitably, the diameter of the nanoparticle (measured by DLS) may be about 20-300 nm or about 20-150 nm or about 20-100 nm or about 20-50 nm or about 30-300 nm or about 50-150 nm or about 80-125 nm or about 90-110 nm. DLS may be performed according to ISO 22412:2017 or a similar technique.

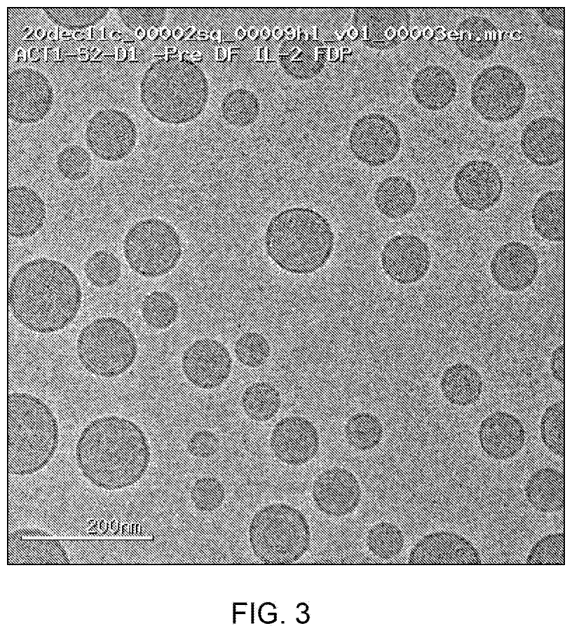

Advantageously, the nanoparticle may be spherical or substantially spherical, and/or may be unilamellar. Exemplary nanoparticles in accordance with one aspect of the disclosure, viewed under Cryo transmission electron microscopy (cryo TEM) may be seen in FIG. 3. Here, it can be seen that the majority of the nanoparticles are spherical and unilamellar; and that the interior of the nanoparticles is more dense than the surrounding buffer, consistent with the loading of imiquimod, HP-β-CD, and PEG 4000 polymer in the interior of the nanoparticle.

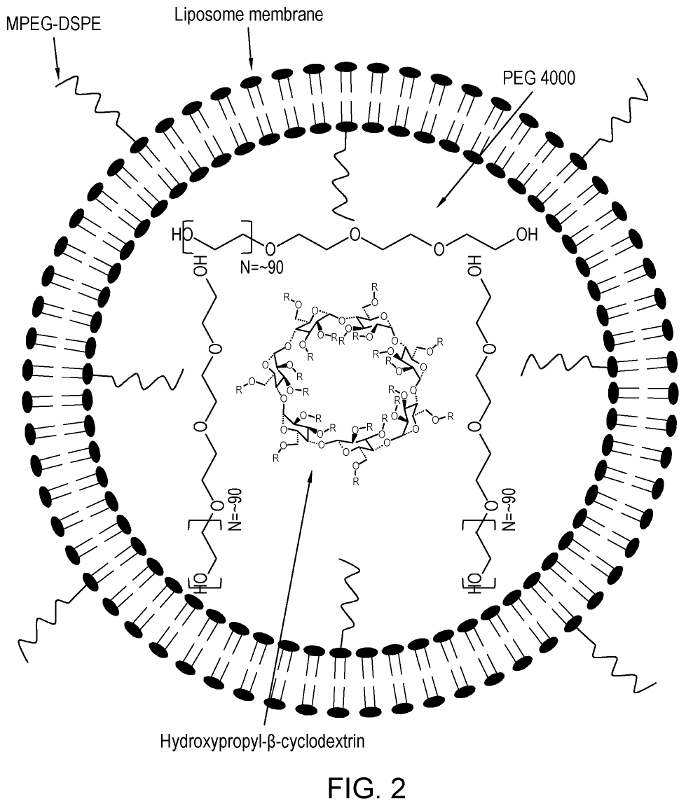

An exemplary nanoparticle in accordance with one aspect of the present disclosure is illustrated in FIG. 2. As shown in this Figure, the exemplary nanoparticle has an outer liposomal shell formed from a lipid bilayer which includes mPEG-DSPE, where mPEG chains are displayed on the outer and inner surfaces of the liposomal shell. The inner core of the nanoparticle comprises a PEG 4000 hydrogel and hydroxypropyl-β-cyclodextrin host molecules. The host molecules reversibly associate with imiquimod molecules in the inner core of the nanoparticle (not shown). IL-2 is loaded into the nanoparticle, and is reversibly attached to or in the outer liposomal shell of the nanoparticle and/or is disposed wholly or partly within the inner core of the nanoparticle (not shown).

According to a further aspect, the present disclosure provides a nanoparticulate composition comprising a plurality of nanoparticles in accordance with this disclosure. The composition may be suitable for therapeutic administration, particularly parenteral administration; and may be sterile. The composition may suitably comprise an aqueous solution, dispersion or suspension of the nanoparticles.

The composition may be buffered to a pH of at least about 6.5, or at least about 7; and preferably no more than about pH 9 or about pH 8.5. Suitably, the composition may be buffered to a pH which is suitably between about pH 6.5-9 or between about pH 6.5-8.5 or between about pH 6.5-8 or between about pH 7-9 or between about pH 7-8.5 or between about pH 7-8 or between about pH 7.5-9. This is beneficial and desirable when it comes to therapeutic use of the composition, where the composition should ideally be buffered to a pH which is close to physiological pH (pH 7.4). Buffering of the composition may be effected using any suitable and acceptable buffering agents, such as citric acid/sodium citrate.

The composition may, suitably, be substantially free of unencapsulated imiquimod. This will help to avoid unwanted precipitation of imiquimod in or from the composition.

In some embodiments, the composition may suitably comprise about 1-100 μg/ml of a protein cytokine such as IL-2. In some embodiments, the composition may comprise about 5-50 μg/ml of protein cytokine such as IL-2 or about 10-40 μg/ml of protein cytokine such as IL-2 or about 20-30 μg/ml of protein cytokine such as IL-2. The composition may comprise at least about 1 μg/ml of protein cytokine such as IL-2, or at least about 5 μg/ml of protein cytokine such as IL-2, or at least about 10 μg/ml of protein cytokine such as IL-2, or at least about 15 μg/ml of protein cytokine such as IL-2. The composition may comprise no more than about 80 μg/ml of protein cytokine such as IL-2 or no more than about 70 μg/ml of protein cytokine such as IL-2 or no more than about 60 μg/ml of protein cytokine such as IL-2 or no more than about 50 μg/ml of protein cytokine such as IL-2 or no more than about 40 μg/ml of protein cytokine such as IL-2 or no more than about 30 μg/ml of protein cytokine such as IL-2.

The composition may comprise about 0.5-50 μg/ml of imiquimod. In some embodiments, the composition may comprise about 1-30 μg/ml of imiquimod or about 5-25 μg/ml of imiquimod. The composition may comprise at least about 1 μg/ml of imiquimod, or at least about 3 μg/ml of imiquimod, or at least about 5 μg/ml of imiquimod. The composition may comprise no more than about 20 μg/ml of imiquimod or no more than about 15 μg/ml of imiquimod or no more than about 13 μg/ml of imiquimod.

In some favoured embodiments, the composition may comprise about 20-30 μg/ml of a protein cytokine such as IL-2 and about 5-15 μg/ml of imiquimod. The composition may comprise about 25 μg/ml of protein cytokine such as IL-2 and about 10 μg/ml of imiquimod.

The composition may comprise about 1-100 mg/ml of lipids. In some embodiments, the composition may comprise about 5-50 mg/ml of lipids or about 10-40 mg/ml of lipids or about 20-30 mg/ml of lipids. The composition may comprise at least about 5 mg/ml of lipids or at least about 10 mg/ml of lipids or at least about 15 mg/ml of lipids or at least about 20 mg/ml of lipids. The composition may comprise no more than about 50 mg/ml of lipids or no more than about 40 mg/ml of lipids or no more than about 30 mg/ml of lipids or no more than about 25 mg/ml of lipids.

The average size of the nanoparticles in the composition, measured by DLS, may suitably be no more than about 300 nm, or no more than about 200 nm, or no more than about 150 nm, or no more than about 130 nm, or no more than about 120 nm. Here, the average size of the nanoparticles in the composition may refer to the mean diameter of nanoparticles in the composition, or may refer to the median particle diameter D50 of the nanoparticles in the composition. In some embodiments, the average size of the nanoparticles in the composition may be at least about 15 nm or at least about 20 nm or at least about 30 nm or at least about 50 nm. In some embodiments, the average size of the nanoparticles in the composition and/or the size range of the nanoparticles in the composition may be about 20-300 nm or about 20-150 nm or about 20-100 nm or about 20-50 nm or about 30-300 nm or about 50-150 nm or about 80-125 nm or about 90-110 nm.

Nanoparticles in accordance with the present disclosure are typically biodegradable, and may advantageously allow for sustained release of imiquimod and, optionally, protein cytokine when the nanoparticles are delivered to a therapeutic site for localised action. This approach permits, inter alia, the administration of imiquimod and IL-2 for combined therapeutic action, in a way which may reduce systemic exposure and associated toxicities, improve the pharmacokinetics, and/or provide therapeutic benefit at doses substantially below the approved therapeutic doses of imiquimod and IL-2.

The inventors report that nanoparticles and compositions according to the present disclosure may provide effective immunotherapy, including effective anti-tumor immunotherapy, through the release and action of imiquimod and, optionally, IL-2 in vivo, for example locally at a tumor site. Imiquimod and IL-2 are well known as active agents, and are well understood by oncologists and immunotherapists for individual and combination therapy. Clinical trials have previously been conducted to evaluate combined therapy using IL-2 injected directly into tumors, with imiquimod applied as a cream to the skin—see Table 3 below:

TABLE 3

| | Clinical | No | IL-2 Dose Ranges | | | |
|---|---|---|---|---|---|---|
| Reference | Phase | Patients | and Imiquimod | Admin | Efficacy | Toxicity |
| Green, | II | 13 | 3.6 million IU IL-2 | 3 × | 41% CR | All Grade |
| Bodman- | | | intralesionally/subcutaneously | weekly | and 9% | 1 or 2 |
| Smith, et | | | And 5% Imiquimod cream | for 15- | PR | |
| al., 2007 | | | every other day for length of | 53 weeks | | |
| | | | trial | | | |
| Shi, Tran, | II | 11 | Test dose 3-7 million IU, | Biweekly | CR | Generally |
| et al., | | | then escalate to 15-22 million | for 4-6 | 100%. | well |
| 2015 | | | IU | weeks | | tolerated. |
| | | | And 5% Imiquimod cream | | | Treatment |
| | | | administered daily, then | | | stopped |
| | | | reduce to 5 x weekly. Post | | | in one |
| | | | IL-2, administered 7 days a | | | patient |
| | | | month for a year | | | due to |
| | | | | | | urticaria. |
| Ogawa, | II | 4 | 10 million IU | Biweekly | CR in | Grade 1 |
| Luxardi, | | | And 5% Imiquimod cream | for 4 | 100% | and |
| et al., | | | | cycles | | Grade 2 |
| 2018 | | | | | | |

CR = complete response; PR = partial response

As seen in Table 3, these trials showed response rates of 41-100%. The mechanism of action of the combination therapy is believed to be based on the immune-stimulatory effects of IL-2, supplemented and enhanced by activation of the innate immune system by imiquimod.

Several findings support the hypothesis that imiquimod can synergistically enhance the activity of IL-2. First, the imidazoquinoline TLR7/8 agonist S28690, an active structural analogue of imiquimod (R-837) has been found to increase expression of CD25 (IL-2 receptor) on CLL cells, thereby enhancing the response of these cells to IL-2 signaling (Tomic et al, J. Immunol. Mar. 15, 2006, 176 (6) 3830-3839). Second, IL-2 induces T cells to differentiate into T effector memory cells while imiquimod enhances the cytotoxic activity of the same cell population (Fiorenza et al, J. Immunol. Dec. 15, 2012, 189 (12) 5622-5631). Third, while IL-2 promotes the growth and differentiation of naïve B cells, TLR7 agonists including resiquimod synergistically enhance IgM and IgG production by IL-2 stimulated B cells (Glaum et al, J. Allergy Clin. Immunol. 2009 January; 123(1):224-230). Additionally, the potential for imiquimod to promote an M1 Hot TAM phenotype is likely to augment the adaptive antitumor responses as reported in Garrido-Martin et al, 2020 J. Immunother. Cancer 8(2).

Nanoparticles and compositions according to the present disclosure may therefore be effective for stimulating or enhancing a therapeutic or protective immune response in a patient. This therapeutic or protective immune response may include the activation or expansion of specific immune cell populations, such as Natural Killer (NK) cells, T cells and/or M1 tumor-destroying macrophages. The therapeutic or protective immune response may be effective to enhance immunosurveillance; that is, recognition and defence against pathogens, allogenic antigens and/or tumor cells. The therapeutic or protective immune response may be effective to reduce local immunosuppression in a tumor microenvironment. The nanoparticles and compositions of the present disclosure may accordingly be effective for the treatment of various medical conditions, including cancer.

According to a further aspect, therefore, the present disclosure provides a method of stimulating or enhancing a therapeutic or protective immune response in a patient, comprising the step of administering a composition in accordance with the present disclosure to a patient in need thereof. The present disclosure provides a method of treating a proliferative disorder such as cancer, comprising the step of administering a composition in accordance with the present disclosure to a patient in need thereof. The cancer may be metastatic cancer. The proliferative disorder may, for example, be melanoma or metastatic melanoma, and/or basal cell carcinoma or metastatic basal cell carcinoma, and/or renal cell carcinoma or metastatic renal cell carcinoma, and/or cutaneous squamous cell carcinoma, and/or Merkel cell carcinoma, and/or extramammary Paget's disease, and/or head and neck cancer or metastatic head and neck cancer. The cancer may be pancreatic cancer, and/or bladder cancer, and/or breast cancer, and/or prostate cancer, and/or lymphoma, and/or liver cancer, and/or colorectal cancer, and/or uveal melanoma, and/or esophogeal cancer, and/or lung cancer, and/or metastases of any of these. The method may involve administering or delivering or allowing for delivery of the nanoparticles in the composition to a lesion such as a tumor. The lesion may, for example, be a primary or metastatic tumor, including a tumor in any of the cancers mentioned herein. The lesion may be a cutaneous metastasis of melanoma, particularly a cutaneous, subcutaneous and/or clinically palpable metastasis in advanced metastatic melanoma, such as a cutaneous or subcutaneous metastatic lesion in a patient with advanced malignant melanoma who has exhausted all standard therapies.

In some embodiments of this aspect of the disclosure, the patient may be a subject whose immune system is partially or completely compromised or suppressed. Immunosuppression refers to a reduction in the activation or efficacy of the immune system. Immunosuppression can arise as a result of or in the course of a disease or condition that impairs immune function, such as AIDS or lymphoma; or as a result of surgery, trauma or injury affecting the spleen or other organs involved in immunity; or it can be medically induced by the action of immunosuppressive drugs, such as anti-rejection medications which are usually administered to a patient following an organ transplant, in order to reduce the risk of organ rejection.

In particular embodiments, therefore, the present disclosure provides a method for stimulating or enhancing a therapeutic or protective immune response or for treating cancer in a patient who is immunosuppressed, optionally a patient who is immunosuppressed by the action or effect of medication that is or has been administered to the patient following an organ transplant; which method comprises the step of administering to the patient a composition in accordance with this disclosure. Suitably, the composition may be locally administered to a tumor, lesion or cancer site in the patient. As referenced above, a recent case study by Vidovic et al (Frontiers in Immunology. 2021 12:1957-1967) reports that locally administered imiquimod and IL-2 is effective for clearing several lesions of high grade squamous cell cancer in a multi-organ transplant patient, with no signs of organ rejection. The present disclosure accordingly provides a further convenient, safe and effective method for treating cancer, particularly skin cancer, in immunosuppressed transplant recipients.

In these and all embodiments of this aspect of the disclosure, the composition may be administered parenterally, for example, by injection or infusion. The composition may be administered intravenously, peritoneally, intrathecally, intravesically, cutaneously or subcutaneously. The composition may be administered systemically, for example by intravenous injection or infusion; or may be administered locally, for example by injection into a cancer site or lesion or the immediate locality of a cancer site or lesion, such as a tumor. Localised administration directly to the tumor or into the peritumoral area may have certain advantages over systemic administration. Notably, localised administration means that systemic exposure to the active agents is minimised, the RES and tumor vasculature barriers are by-passed, and local/regional spread of the cancer can be more effectively addressed.

The composition may be administered locally into or near to a target lesion such as a tumor for treatment of the target lesion and/or for treatment of distal lesions, such as distal or metastatic tumors, by way of an abscopal effect. Experimental results published by Nektar Therapeutics demonstrate an abscopal effect on distal tumors when a TLR7/8 agonist NKTR-262 is locally administered to a target tumor by injection in combination with systemic administration of a pegylated IL-2 prodrug, NKTR-214; see Kivimae et al, “Comprehensive Antitumor Immune Activation by a Novel TLR 7/8 Targeting Agent NKTR-12262 Combined With CD122-Biased Immunostimulatory Cytokine NKTR-214” and Rolig et al, “NKTR-214 (CD122-biased agonist) and NKTR-262 (TLR7/8 agonist) combination treatment pairs local innate immune activation with systemic CD8+ T cell expansion to enhance anti-tumor immunity”, both presented at American Association for Cancer Research 2018 Annual Meeting, Apr. 14-18, 2018.

The composition may, alternatively, be administered systemically by intravenous infusion for enhanced treatment of lesions such as tumors by way of an abscopal effect. It is known that systemic intravenous administration of nanoparticulate therapeutic compositions does not always yield a uniform distribution of the drug across all tumors in the body; but may result in certain tumors in a patient receiving lower-than-average amounts of the administered drug (see Lee et al, Clin. Cancer Res. 23(15) Aug. 1, 2017 4190-4202). The present disclosure provides for effective systemic treatment of tumors which receive lower-than-average amounts of the disclosed composition upon systemic administration, by way of an abscopal effect.

The composition may be administered to a patient in need thereof in one or more spaced doses. Thus, the method may involve administering to the patient one, two, three, four or five spaced doses of the composition per day, or administering to the patient one, two, three, four, five or six spaced doses of the composition per week. In some embodiments, the method may involve administering to the patient one or two spaced doses of the composition per week. Weekly or bi-weekly administration is supported by the sustained release of the active agents (e.g. imiquimod and IL-2) from the composition. In particular, the inventors report that nanoparticle formulations as disclosed herein may provide controlled/sustained release of the active agents over a period of several days. The exemplified starting dose of the formulation contains ˜400,000 IU of IL-2 and 10 μg of imiquimod. These amounts of API have demonstrated anti-tumor activity in animal models (Broomfield, van der Most, et al., 2009; Den Otter, Jacobs, et al., 2008) and are significantly lower than doses shown to be well tolerated in patients. The present disclosure therefore allows for a reduction in systemic exposure and reduced toxicities.

The method may involve administering each dose of the composition as a single injection or infusion or as a plurality of simultaneous or consecutive injections or infusions. Each dose may, for example, comprise a single injection or a plurality of simultaneous or consecutive injections, delivered to a cancer site, such as into or into the immediate locality of one lesion, or into or into the immediate locality of a plurality of lesions. Alternatively, each dose may comprise a single intravenous injection or infusion.

In embodiments where the composition is administered by injection into a lesion or into the immediate locality of a lesion, each dose may suitably comprise about 0.25 μg-250 μg of imiquimod; suitably, each dose or each injection may comprise about 0.25 μg-50 μg of imiquimod. Additionally or alternatively, each dose may comprise about 0.5 mg to 500 mg of lipids; suitably, each dose or each injection may comprise from about 0.5 mg-50 mg of lipids. The volume of each dose may suitably be about 0.025 ml to 25 ml; suitably, the volume of each dose or each injection may be about 0.025 ml to 2.5 ml. Additionally or alternatively, each dose may comprise about 10,000 IU to about 10,000,000 IU of protein cytokine such as IL-2, or from about 0.625 μg to about 625 μg of protein cytokine such as IL-2; suitably, each dose or each injection may comprise about 10,000 IU-1,000,000 IU of protein cytokine such as IL-2 or about 0.625 μg-62.5 μg of protein cytokine such as IL-2.

In embodiments where the composition is administered by intravenous infusion, each dose may suitably comprise about 50 μg to 5000 μg of imiquimod; for example, about 50 μg to 1000 μg of imiquimod, or about 1000-5000 μg of imiquimod. Additionally or alternatively, each dose may suitably comprise about 100 mg to 10,000 mg of lipids; for example, about 100-1000 mg of lipids or about 1000-10,000 mg of lipids. The volume of each infused dose may suitably be 5 ml to 500 ml; for example, 5-100 ml, or 100-500 ml. Additionally or alternatively, each dose may suitably comprise about 2,000,000 IU (125 μg) to 200,000,000 IU (12.5 mg) of protein cytokine such as IL-2; for example, about 125 μg to 2.5 mg of protein cytokine such as IL-2 or 2.5 mg-12.5 mg of protein cytokine such as IL-2.

Nanoparticles according to the present disclosure may be formulated to contain imiquimod and, optionally, protein cytokine such as IL-2, at concentrations sufficient to deliver an effective dose for both intratumoral and intravenous administration. In some embodiments, the maximum dose of the nanoparticles may contain about 100 μg (1.6 million IU) of IL-2, which is approximately 26-fold less than the approved human Proleukin® dose for a 70 kg patient. The maximum dose of the nanoparticles may contain about 40 μg imiquimod, which is approximately 625-fold less than the maximum recommended human dose (MRHD) and 750-fold less than a well tolerated 30 mg subcutaneous dose in healthy human volunteers.

The efficacy of the presently disclosed compositions and nanoparticles allows for the administration of relatively low doses. As shown in Table 4 below, the quantity of excipients administered to a 70 kg patient in an exemplary 4 ml dose of an exemplary nanoparticle composition according to the disclosure (NP) is less than the equivalent excipient dose in marketed parenteral products:

TABLE 4

| | | | Excipient Dose | |
|---|---|---|---|---|
| | | Excipient Dose | from NP (4 mL | |
| | Approved | from Approved | injection and | Safety |
| Excipient | Drug | Drug | 70 kg patient) | Factor |
| Lipids | Doxil | 10 mg/kg | 1.1 mg/kg | 9-fold |
| | | (intravenous) | (subcutaneous) | |
| HP-β-CD | Mitozytrex | 145 mg/kg | 13 mg/kg* | 11-fold |
| | | (intravenous) | (subcutaneous) | |
| PEG | INVEGA | 75 mg | 31 mg* | 2-fold |
| 4000 | SUSTENNA | (intramuscular) | (subcutaneous | |

*The estimated patient dose of PEG 4000 and HP-β-CD from NP are a “worst case estimate” based upon the batch formula, and assuming no removal of unencapsulated PEG 4000 and 2-hydroxypropyl-β-cyclodextrin during the manufacturing process.

In embodiments where the composition is or may be administered by intravenous infusion, the nanoparticles in the composition may advantageously have an average diameter which allows for an enhanced permeability and retention (EPR) effect, where nanoparticles administered via intravenous infusion extravasate from the blood vessels at the tumor and accumulate in the tumor where they release the APIs. To this end, the average (mean or median) diameter of the nanoparticles may be about 20-300 nm or about 20-150 nm, or about 20-100 nm, or about 20-50 nm, or about 30-300 nm, or about 50-150 nm, or about 80-125 nm, or about 80-120 nm, or about 90-110 nm, or about 90-100 nm, or about 100 nm. In these embodiments, the nanoparticles are sized to selectively extravasate in tumor tissues with abnormal vascularity, where they are retained due to the tumor's inefficient lymphatic drainage. The extravasation effect can therefore increase accumulation of the nanoparticles at the tumor site.

Further embraced within the scope of this disclosure are nanoparticles and compositions as disclosed which are suitable for the therapeutic methods disclosed herein; nanoparticles and compositions as disclosed for use in the therapeutic methods disclosed herein; and the use of nanoparticles as disclosed in the manufacture of compositions and medicaments for use in the therapeutic methods disclosed herein.

Aqueous core-lipid shell nanoparticles for drug delivery are known in the art, and have been described in general terms, for example in WO 2013/155487. However, the production of aqueous core-lipid shell nanoparticles comprising imiquimod and, optionally, protein cytokine, as disclosed herein, has not previously been described or suggested and has not proved to be straightforward. Whilst imiquimod can be solubilised in an aqueous environment at acidic pH, acidic pH formulations are not compatible with lipid stability or protein cytokine stability; nor are they suitable for parenterally administered drugs. Because of these issues, it was not obvious that an aqueous core-lipid shell nanoparticle formulation comprising imiquimod and, optionally a protein cytokine such as IL-2, suitable for parenteral administration, could be successfully developed, even applying the known techniques disclosed in WO 2013/155487.

The inventors have nevertheless successfully developed and disclose herein a method for manufacturing a nanoparticle and composition as disclosed, which comprises the sequential steps of
(a) solubilising imiquimod in aqueous solution at a pH buffered to about pH 6 or below; followed by
(b) combining the resulting aqueous solution with lipids to form lipid shell nanoparticles encapsulating imiquimod; followed by
(c) increasing the buffered pH of the formulation to about pH 6.5 or above.

Optionally, step (c) may be followed by
(d) adding a protein cytokine such as IL-2 to the formulation of (c) to load the nanoparticles with the protein cytokine.

This method as developed by the inventors allows for adjustment of the pH during the process in such a manner as to achieve a formulation at neutral (physiological) pH, whilst minimising or avoiding precipitation of unencapsulated imiquimod or IL-2 from the formulation.

Suitably, step (a) of the process may involve solubilising imiquimod at a pH buffered to about pH 4-6, or to about pH 4.5-6, or to about pH 4-5.5, or to about pH 5-6. Step (c) of the process may involve increasing the buffered pH of the formulation to about pH 7 or above, or to about pH 6.5-9, or to about pH 6.5-8.5, or to about pH 6.5-8; or to about pH 7-9; or to about pH 7-8.5; or to about pH 7-8; or to about pH 7.5-9. Step (a) may, for example, involve solubilising imiquimod in aqueous solution in the presence of a hydroxyacid, such as citric acid, tartaric acid, lactic acid, glycolic acid or malic acid. Step (a) may involve solubilising imiquimod in aqueous solution in the presence of a host molecule as disclosed herein, such as a cyclodextrin, in particular HP-β-CD. In particular, step (a) may involve combining imiquimod with a host molecule such as cyclodextrin in solution at a pH buffered to about pH 6 or below, preferably to about pH 4-6, or to about pH 4.5-6, or to about pH 4-5.5, or to about pH 3-5.5, or to about pH 5-6, or to about pH 5.

Step (b) may involve mixing the solution of (a) with lipids as disclosed herein, to form a solution or suspension of multilamellar structures which may or may not be large multilamellar structures; and processing these multilamellar structures to form lipid shell nanoparticles encapsulating imiquimod. The lipids may optionally be solubilised in alcoholic solution. The step of processing the structures may comprise extruding the solution or suspension of multilamellar structures through membranes to form lipid shell nanoparticles encapsulating imiquimod; or may comprise drying the solution or suspension of multilamellar structures to form a film, solubilising the film, and shaking or sonicating the resulting solution to form lipid shell nanoparticles encapsulating imiquimod; or may comprise using microfluidic mixing technologies to form lipid shell nanoparticles encapsulating imiquimod.

In other embodiments, step (b) may involve mixing the solution of (a) with empty liposomes to form lipid shell nanoparticles encapsulating imiquimod. In this case, the empty liposomes may be formed from lipids as disclosed herein.

In some embodiments, the buffered pH of the formulation may be increased in step (c) by known techniques such as diafiltration or buffer exchange. The method may further comprise a step of adding a hydrogel polymer as disclosed herein, such as a PEG 4000 polymer, between step (a) and step (b), or between step (b) and step (c), or between step (c) and step (d), or after step (d).

In some embodiments, the method may further comprise reducing unencapsulated imiquimod from the nanoparticle formulation of (b), after step (b) and before step (c). This may optionally be done by ultracentrifugation or, preferably, by diafiltration with membranes which are sized to retain the nanoparticles but permit the passage of free imiquimod, or by other techniques known in the art.

After completion of steps (a)-(d), the resulting nanoparticle product may be subject to standard processing steps, including concentration adjustment, the addition of suitable excipients, sterilisation etc, prior to being dispensed into a suitable container for storage prior to administration.

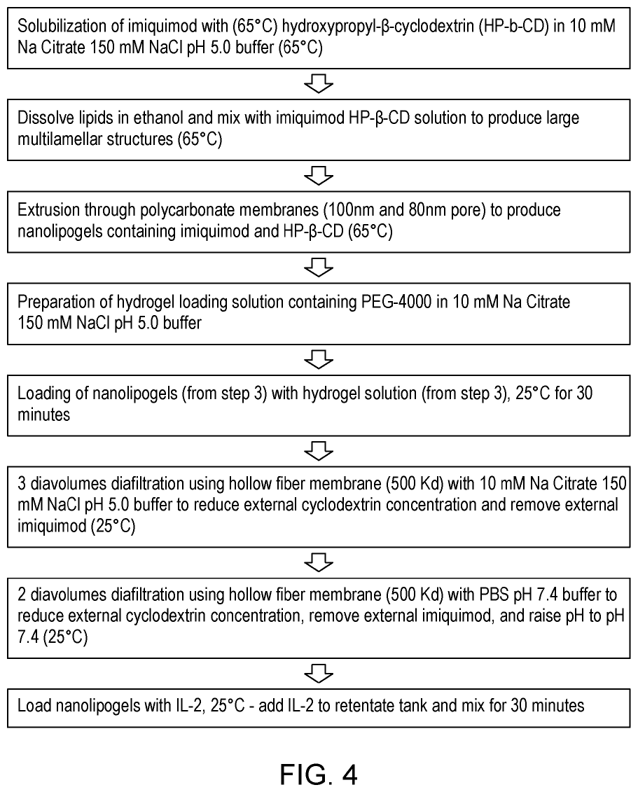

An example of the manufacturing process is illustrated in FIG. 4 and described in Examples 1-3. The process as illustrated and described utilizes acidic pH buffers and HP-β-CD concentrations which are optimized for the incorporation of imiquimod:cyclodextrin into nanoparticles during the extrusion process. The present inventors have recognised that as acidic pH conditions are not compatible with lipid stability and protein cytokine, including IL-2, stability, and are unsuitable for parenterally administered drugs, the ingredients of the nanoparticle should not be exposed to a pH below pH 4 during the manufacturing process, and the pH must be raised to near neutral pH in the final product (and before the optional addition of protein cytokine). Because imiquimod solubility is greatly decreased at near neutral pH, the concentration of “free” unencapsulated imiquimod in the in-process solution must be reduced prior to any increase in pH to prevent precipitation of the “free” unencapsulated imiquimod which would block filters used for dialfiltration and sterile filtration, preventing its removal from the bulk product and preventing successful sterile filtration. The reduction in the concentration of “free” unencapsulated imiquimod can be accomplished by methods such as ultracentrifugation or diafiltration with appropriately sized membranes that retain nanoparticles, but permit the passage of free imiquimod and cyclodextrin into the permeate. Diafiltration is a process that is well suited for clinical and commercial scale drug manufacturing, and may be preferred for this reason. After the concentration of “free” unencapsulated imiquimod in the in-process solution is reduced sufficiently so that it does not precipitate at near neutral pH, the pH can be raised and diafiltration can continue to remove external cyclodextrin and imiquimod, and formulate the product into Drug Product final buffer.

The optional addition of protein cytokine occurs after the pH has been adjusted to near neutral, as disclosed herein. This may be before or after all necessary diafiltration and purification steps have been completed, as described in Example 2.

A batch process performed according to cGMP standards may produce approximately 10 liters of the nanoparticle composition, being a sterile suspension of liposomes containing recombinant human IL-2 and imiquimod at concentrations of 25 μg/mL and 10 μg/mL, respectively; hence containing approximately 100 mg of imiquimod, and 250 mg of IL-2 and 200 g of lipids. This composition may be dispensed into sterilised vessels, ready for administration to a patient by injection or infusion. The vessels may, for example, be vials having a volume of 2-10 ml which are intended for and suitable for intratumoral injection. Alternatively, the vessels may be bottles or pouches having a volume of 10-500 ml which are intended for and suitable for intravenous injection or infusion.

Following are specific examples according to the present disclosure.

### Example 1: Nanoparticle Components

Nanoparticles were produced using a number of raw materials and excipients important for the structure and quality of the drug product. With the exception of cholesterol (see below), all of the raw materials and excipients were either synthetic or derived from plants.

The liposome shell was composed of three components:
N-(carbonyl-) methoxypolyethylene glycol 2000)-1,2-distearoyl-sn-glycero 3-phosphoethanolamine sodium salt (MPEG-DSPE)
fully hydrogenated soy phosphatidylcholine (HSPC), and
cholesterol

The hydrogel core was composed of two components:
2-hydroxypropyl-β-cyclodextrin
polyethylene glycol 4000 hydrogel

These components are all commercially available and are known and well-characterised excipients.

### Example 2: Preparation of Nanoparticles

The steps of a process for production of nanoparticles is illustrated in FIG. 4. Imiquimod was solubilised in aqueous solution at pH 5 in the presence of HP-β-CD. A solution of lipids was produced by dissolving fully hydrogenated soy phosphatidylcholine (HSPC), N-(carbonyl-methoxypolyethylene glycol 2000)-1,2-distearoyl-sn-glycero 3-phosphoethanolamine sodium salt (MPEG-DSPE) and cholesterol in ethanol at a molar ratio of about 1.9:0.1:1). This solution was mixed with the imiquimod solution to form multilamellar structures. These structures were then extruded through membranes with appropriate pore sizes (80 to 100 nm) to produce nanoparticles or “nanolipogels”, which are core-shell structures having a lipid shell encapsulating imiquimod and HP-β-CD.

The nanolipogels were incubated with a solution of PEG-4000 in pH 5 buffer, allowing the PEG-4000 to load into the interior of the nanoparticles.

Diafiltration was carried out to remove external imiquimod and cyclodextrin. Further diafiltration steps were then performed to raise the pH of the formulation to pH 7.4.

The nanoparticles were then incubated with IL-2 in solution at pH 7.4, allowing the IL-2 to load into and onto the nanoparticles.

### Example 3. Preparation of a Drug Product

The nanoparticles of Example 2 were subjected to 4 diavolumes diafiltration using hollow fiber membrane (500 Kd) with 1% trehalose PBS pH 7.4 buffer at 25° C., to further reduce external cyclodextrin concentration and remove external imiquimod, and to add 1% trehalose. This step may, alternatively, be carried out during the production of the nanoparticles according to Example 2, before or after the addition of IL-2.

The concentration of the formulation was then adjusted and the formulation was sterilised to a standard required for therapeutic use. Sterilisation was carried out by filtration, using a 0.2 μm filter.

The formulation is supplied at a single dose concentration containing 25 μg/mL and 10 μg/mL of IL 2 and imiquimod, respectively. It is a sterile, white opaque, liquid. The container closure system consists of a 5 mL clear borosilicate glass vial, a 13 mm synthetic chlorobutyl rubber stopper, and flip-off crimp seal. The composition of the IMP in a 5 ml vial is listed in Table 5 below.

TABLE 5

| | Amount | | |
|---|---|---|---|
| Component | (mg) | Function | Quality Standard |
| Imiquimod | 0.05 | Active Ingredient | USP |
| Interleukin-2 | 0.125 | Active Ingredient | US and European |
| (Proleukin ®) | | | Marketed Product |
| HSPC | 69.3 | Vesicle forming | Manufacturer |
| | | lipid | specifications |
| MPEG-DSPE | 12.6 | Vesicle forming | Manufacturer |
| | | lipid | specifications |
| Cholesterol | 17.7 | Vesicle forming | Multi (USP/Ph. |
| | | lipid | Eur./BP/JP) |
| PEG 4000 | 39* | Drug trapping | Manufacturer |
| | | agent | specifications |
| 2-hydroxypropyl- | 1170* | Drug trapping | USP/Ph. Eur. |
| β-cyclodextrin | | agent | |
| Sodium | 13 | Buffer | Multi (USP/Ph. |
| phosphate | | | Eur./BP/JP) |
| Sodium chloride | 17 | Isotonicity | Multi (USP/Ph. |
| | | | Eur./BP/JP) |
| Trehalose | 18 | Isotonicity | Multi (USP/Ph. |
| | | | Eur./BP/JP) |
| Water for | 5,000 | Solvent | Multi (USP/Ph. |
| injection | | | Eur./JP) |
| Sodium | as required | pH adjustor | Multi (USP/Ph. |
| hydroxide | | | Eur./BP/JP) |
| Hydrochloric acid | as required | pH adjustor | NF/Multi (USP/Ph. |
| | | | Eur./BP/JP) |

*The concentrations of the PEG 4000 and 2-hydroxypropyl-β-cyclodextrin are the maximum possible concentrations based upon the batch formula.

### Example 4: Drug Product Properties

A Cryo-TEM image of the nanoparticles is shown in FIG. 3. Cryo transmission electron microscopy (cryo TEM) demonstrates that the majority of ACT-1 liposomes are spherical unilamellar liposomes. The cryo TEM images also provide information related to the state of the encapsulated drug and the internal environment of the ACT-1 liposomes. The ACT-1 liposomes have interiors that are more dense than the surrounding buffer, consistent with the loading of the liposomes with imiquimod, HP-β-CD, and the PEG 4000 polymer in the interior of the liposome.

### Example 5: Non-Clinical Toxicity and Anti-Tumor Efficacy Studies

This example references studies which indicate that the APIs utilised in the presently disclosed nanoparticles are well tolerated in animals at doses greater than the clinical doses disclosed herein; and studies which demonstrate anti-tumor activity of imiquimod and IL-2 doses similar to the amounts of imiquimod and IL-2 delivered by way of the presently disclosed nanoparticles.

Human interleukin-2 (IL-2) was administered subcutaneously to rats at doses of 0.3-10 mg/kg/day in a range finding study and 0.03-0.3 mg/kg/day in a 4-week toxicity study (Wolfgang, McCabe, et al., 1998). Treatment-related effects were assessed by hematology, clinical chemistry, anti-rIL-2 antibody production, and gross and histopathologic evaluations. Doses of 1 mg/kg/day or above were not tolerated, resulting in death or moribund termination by Day 7. Slight decreases in red blood cell counts (including hematocrit and hemoglobin) were observed at greater or equal to 0.1 mg/kg/day. White blood cells counts increased in a dose-dependent manner; increases were primarily due to increases in lymphocytes and eosinophils. Hepatic abnormalities, including increases in aspartate aminotransferase and bilirubin, were noted at 0.3 mg/kg/day. Histologic findings were evident primarily in the spleen, liver, lung, and injection sites, with dose-related increases in inflammatory cell foci/infiltrates noted in these sites. Findings in the liver also included biliary hyperplasia, hepatocellular degeneration, necrosis, vascular mural thickening in the portal triads, and fibrosis. Red and white pulp hyperplasia and capsular fibrosis occurred in the spleen. Most clinical and histopathologic findings were reversible within 4 weeks after termination of treatment.

The anti-tumor activity of IL 2 has been investigated in many mouse models of tumors and shown to be effective when administered intralesionally at doses ranging from 300 to 300,000 IU (Den Otter, Jacobs, et al., 2008).

The FDA Pharmacology/Toxicology review for Zyclara (imiquimod) cream 3.75% (Application Number 201153) describes a number of animal studies of the toxicology, pharmacokinetics, and metabolism of imiquimod in different species, and the findings from these studies include:
The subcutaneous lethal dose of a single subcutaneous dose in rats was 20 mg/kg.
Intravenous doses of 0.5 to 5.0 mg/kg imiquimod produced cardiac stimulation, central nervous system stimulation and some evidence of autonomic nervous system inhibition in dogs.
Intravenous doses of 0.5, 1 and 2 mg/kg/day imiquimod were administered to pregnant female rabbits during the period of organogenesis (gestational Days 6-18). No treatment related effects on embryofetal toxicity or teratogenicity were noted at 2 mg/kg/day.

The EMA's toxicology review of Aldara (imiquimod) 5% cream (https://www.ema.europa.eu/en/documents/scientific-discussion/aldara-epar-scientific-discussion_en.pdf) describes a number of animal studies of the toxicology, pharmacokinetics, and metabolism of imiquimod. It was reported that overall, “the toxicology program indicates a high degree of safety with no target organ toxicity other than that attributed to exaggerated pharmacological activity. Imiquimod did not affect fertility and it was neither teratogenic nor genotoxic. In carcinogenicity study in mice there was no increase in the incidence of tumors or non-neoplastic lesions as the result of dermal exposure to imiquimod”. Specific findings from these studies include:
Single dose toxicity of imiquimod studied in mice, rats, and monkeys indicated a high degree of safety. Adverse effects were limited to the central nervous system resulting in a number of clinical signs, usually convulsions, prior to death.
In two dermal toxicity studies in rabbits with doses of 2000 and 5000 mg/kg under occlusion there were no deaths and no signs of toxicity other than mild transient erythema at the application site.
Repeated dose toxicity of imiquimod after oral administration in rats and monkeys up to 6 months showed the only adverse effects apart from slight effects on body weight and food consumption were considered to be the result of exaggerated pharmacological activity, i.e., hyperplasia of B and T cell lymphoid tissue, increased number of plasma cells, enlargement of spleen and lymph nodes, Kupffer cell hyperplasia, mononuclear/macrophage cell accumulation or proliferation. There were no other target organs and a no observed adverse effect level (NOAEL) of 3 mg/kg was established in both species.

The anti-tumor activity of imiquimod has been investigated in animal models of tumors with varying degrees of efficacy. Broomfield, van der Most, et al., 2009 screened the antitumor activity of imiquimod using a number of dosing regimens (q2d×3, q2d×6, q1d×3) and doses (1 μg to 1 mg) delivered through various routes (intraperitoneal, subcutaneous, intravenous, topical, peritumoral, and intratumoral). Only locally (intratumoral) delivered imiquimod consistently and significantly retarded the growth of tumor in mouse models that included mesothelioma, renal cell carcinoma and 4T1 breast cancer. Maximum inhibition of tumor growth resulted in a >50% increase in survival time using 50 μL injections of 50 μg/mL imiquimod (p<0.05) and was not associated with overt signs of toxicity (such as weight loss and ruffled fur). Finally, mice cured mice resisted tumor re-challenge indicating a memory T cell response was obtained.

The API doses of the nanoparticles disclosed herein are tens to hundreds fold below the current dose levels of approved drugs or the MRHD based on numerous animal studies or a well tolerated 30 mg subcutaneous dose in healthy human volunteers (Soria, Myhre, et al., 2000). See also Tables 6a and 6b below, which compare the quantities of IL-2 and imiquimod in a proposed 1 ml starting dose and a 4 ml maximum feasible dose of the disclosed nanoparticles (NP) with the approved safe doses of Proleukin® (IL-2) and Aldara® (imiquimod). Further, PK data from published studies indicates minimal systemic exposure when L (2 or imiquimod is injected subcutaneously into patients.

TABLE 6a

| | | | | | Imiquimod | Imiquimod |
|---|---|---|---|---|---|---|
| IL-2 dose | IL-2 Dose | | | Imiquimod | Safety | Safety Factor |
| (μg) in | (μg/kg) in | | | Dose (μg) | Factor for | for 30 mg |
| 1 mL | 1 mL | | Proleukin | in 1 mL | 25 mg | Human |
| Volume | Volume | | Safety | Volume | Aldara | Subcutaneous |
| of NP | NP | IL-2 in | Factor | NP | MRHD mg | Dose (Soria, |
| starting | starting | Single Dose | (Single | starting | (Single | Myhre, et al., |
| dose | dose | of Proleukin | Dose) | dose | Dose) | 2000) |
| 25 μg | 0.36 μg/kg | ei37 μg/kg | 104-fold | 10 μg | 2500-fold | 3000-fold |
| | (5700 IU/kg) | (600,000 | | | | |
| | (subcutaneous) | IU/kg) | | | | |
| | | (intravenous) | | | | |

MRHD = maximum recommended human dose

TABLE 6b

| | | | | | Imiquimod | Imiquimod |
|---|---|---|---|---|---|---|
| | | | | | Safety | Safety Factor |
| IL-2 dose | IL-2 Dose | | | | Factor for | for 30 mg |
| (μg) in | (μg/kg) in | | Proleukin | Imiquimod | 25 mg | Human |
| 4 mL | 4 mL Volume | IL-2 in | Safety | Dose (μg) | Aldara | Subcutaneous |
| Volume | MFD of NP | Single | Factor | in 4 mL | MRHD mg | Dose (Soria, |
| MFD of | for 70 kg | Dose of | (Single | Volume | (Single | Myhre, et al., |
| NP | Patient | Proleukin | Dose) | MFD of NP | Dose) | 2000) |
| 100 μg | 1.4 μg/kg | 37 μg/kg | 26-fold | 40 μg | 625-fold | 750-fold |
| | (22,880 IU/kg) | (600,000 | | | | |
| | (subcutaneous) | IU/kg) | | | | |
| | | (intravenous) | | | | |

MFD = maximum feasible dose; MRHD = maximum recommended human dose

### Example 6. In Vitro Release Assays

In vitro release assays (IVRA) have been developed for imiquimod and will be developed for IL-2.

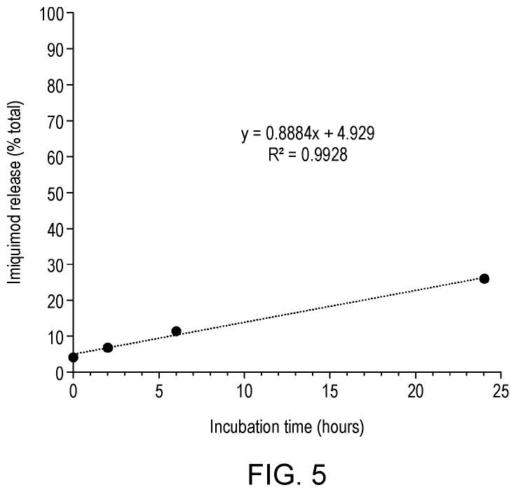

Results of the IVRA are illustrated in FIG. 5. Here, nanoparticles produced according to Examples 1-3 were diluted with an equal volume of PBS buffer and incubated with gentle shaking at 37° C. Samples were removed at 0, 4, 6 and 24 hours and immediately processed using a 300 kd filter to obtain the released free (non-encapsulated) imiquimod in the filtrate. The concentration of imiquimod in the filtrate was determined using a reverse phase HPLC assay.

The results show that imiquimod is released from the nanolipogels at a linear rate over time, with approximately 25% of the drug released after 24 hours. These in vitro results evidence the ability of the nanolipogels to provide sustained delivery of therapeutically effective quantities of imiquimod in the tumor.

1. A nanoparticle for co-delivery and, optionally, sustained release of an imidazoquinoline and a protein cytokine such as IL-2, comprising an outer lipid shell and an inner aqueous core encapsulated within the outer lipid shell;
wherein the imidazoquinoline is an active TLR7/8 ligand having the molecular structure 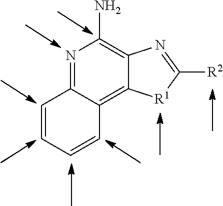
where R¹ is N and R² is H or C, and where the imidazoquinoline is unsubstituted or is substituted at one or more of the indicated addition points with one or more substituents, which substituents are independently selected from branched, linear or cyclic alkyl, alkenyl, alcohol, alkylamine, alkoxy or alkoxyalkyl groups, in particular, C₁₋₁₀ alkyl, alkenyl, alcohol, alkylamine, alkoxy or alkoxyalkyl groups, or hydroxyl groups, or amine groups, or N—(C₁₋₁₀ alkyl)methanesulphonamide groups;
wherein the inner aqueous core of the nanoparticle comprises the imidazoquinoline;
and wherein the nanoparticle further comprises a protein cytokine such as IL-2 releasably attached to, associated with and/or encapsulated within the lipid shell.

2. The nanoparticle of claim 1, wherein the inner aqueous core further comprises a host molecule that is capable of reversibly forming a complex with the imidazoquinoline, wherein the host molecule is or includes a cyclodextrin; preferably a cyclodextrin selected from α-cyclodextrin; β-cyclodextrin; γ-cyclodextrin; methyl α-cyclodextrin; methyl β-cyclodextrin; methyl γ-cyclodextrin; ethyl β-cyclodextrin; butyl α-cyclodextrin; butyl β-cyclodextrin; butyl γ-cyclodextrin; pentyl γ-cyclodextrin; hydroxyethyl β-cyclodextrin; hydroxyethyl γ-cyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl α-cyclodextrin; acetyl β-cyclodextrin; acetyl γ-cyclodextrin; propionyl β-cyclodextrin; butyryl β-cyclodextrin; succinyl α-cyclodextrin; succinyl β-cyclodextrin; succinyl γ-cyclodextrin; benzoyl β-cyclodextrin; palmityl β-cyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl β-cyclodextrin; phosphate ester α-cyclodextrin; phosphate ester β-cyclodextrin; phosphate ester γ-cyclodextrin; 3-trimethylammonium-2-hydroxypropyl β-cyclodextrin; sulfobutyl ether β-cyclodextrin; carboxymethyl α-cyclodextrin; carboxymethyl β-cyclodextrin; carboxymethyl γ-cyclodextrin, and combinations thereof; advantageously, wherein the cyclodextrin is or includes 2-hydroxypropyl-β-cyclodextrin.

3-4. (canceled)

5. The nanoparticle of claim 1, wherein the imidazoquinoline is imiquimod (R-837) or an active structural analogue of imiquimod (R-837), such as resiquimod, gardiquimod, S28690, 852-A, 854A, CL075 or CL097.

6. The nanoparticle claim 1, wherein the inner aqueous core comprises a hydrogel polymer selected from poly(lactic acid), poly(glycolic acid), poly(lactic acid-co-glycolic acids), polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); poly(glycolide-co-caprolactones); polycarbonates; polyamides, polypeptides, and poly(amino acids); polyesteramides; other biocompatible polyesters; poly(dioxanones); poly(alkylene alkylates); hydrophilic polyethers; polyurethanes; polyetheresters; polyacetals; polycyanoacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), polyvinyl alcohols, polyvinylpyrrolidone; poly(alkylene oxides); celluloses, polyacrylic acids, albumin, collagen, gelatin, prolamines, polysaccharides, derivatives, copolymers, and blends thereof.

7. The nanoparticle of claim 1, which nanoparticle is decorated with one or more targeting agents displayed on the outer surface of the lipid shell, such as an antibody, peptide, protein, aptamer or small molecule targeting agent.

8. A nanoparticulate composition comprising an aqueous solution, dispersion or suspension of nanoparticles of claim 1, wherein the composition is buffered to a pH of at least about 6.5, preferably at least about 7; suitably between about pH 6.5-9 or between about pH 6.5-8.5 or between about pH 6.5-8 or between about pH 7-9 or between about pH 7-8.5 or between about pH 7-8 or between about pH 7.5-9.

9-10. (canceled)

11. A method for stimulating or enhancing a therapeutic or protective immune response or for treating a proliferative disorder such as cancer in a patient, comprising a step of administering the composition of claim 8 to the patient.

12. The method of claim 11, wherein the patient is a subject whose immune system is partially or completely suppressed; optionally, wherein the patient is a subject who suffers from a disease or condition that impairs immune function, such as AIDS or lymphoma; or a subject who receives or has received one or more doses of an immunosuppressive drug, for example following an organ transplant.

13. The method of claim 11, wherein the proliferative disorder includes melanoma or metastatic melanoma, and/or basal cell carcinoma or metastatic basal cell carcinoma, and/or cutaneous squamous cell carcinoma, and/or Merkel cell carcinoma, and/or breast cancer, and/or extramammary Paget's disease, and/or renal cell carcinoma or metastatic renal cell carcinoma, and/or head and neck cancer or metastatic head and neck cancer, and/or wherein the cancer includes pancreatic cancer, or bladder cancer, or prostate cancer, or lymphoma, or liver cancer, or colorectal cancer, or uveal melanoma, or esophogeal cancer, or lung cancer, or metastases of any of these.

14. The method of claim 11, wherein the composition is administered parenterally, optionally by injection or infusion.

15. The method of claim 11, which comprises administering to the patient one, two, three, four or five spaced doses of the composition per day, or administering to the patient one, two, three, four, five or six spaced doses of the composition per week; preferably wherein the method comprises administering to the patient one or two spaced doses of the composition per week.

16. The method of claim 15, which comprises administering each dose of the composition as a single injection or infusion or as a plurality of simultaneous or consecutive injections or infusions.

17. The method of claim 11, which comprises administering the composition systemically, or administering the composition locally to or near to a cancer site or a lesion such as a tumor.

18-19. (canceled)

20. A method for manufacturing the nanoparticle of claim 1, comprising the sequential steps of:
(a) solubilising the imidazoquinoline in aqueous solution at a pH buffered to about pH 6 or below; preferably to about pH 4-6, or to about pH 4.5-6, or to about pH 4-5.5, or to about pH 5-6; followed by
(b) combining the resulting aqueous solution with lipids to form lipid shell nanoparticles encapsulating the imidazoquinoline; followed by
(c) increasing the buffered pH of the formulation to about pH 6.5 or above, or to about pH 7 or above, or to about pH 6.5-9, or to about pH 6.5-8.5, or to about pH 6.5-8; or to about pH 7-9, or to about pH 7-8.5 or to about pH 7-8 or to about pH 7.5-9.

21. The method of claim 20, wherein step (c) is followed by
(d) adding a protein cytokine such as IL-2 to the formulation of (c) to load the nanoparticles with the protein cytokine.

22. The method of claim 20, wherein step (a) comprises solubilising the imidazoquinoline with a host molecule such as a cyclodextrin in aqueous solution at a pH buffered to about pH 6 or below, preferably to about pH 4-6, or to about pH 4.5-6, or to about pH 4-5.5, or to about pH 5-6.

23. The method of claim 20, wherein step (b) comprises mixing the solution of (a) with lipids, which are optionally solubilised in alcoholic solution, to form a solution or suspension of multilamellar structures, and processing these structures to form lipid shell nanoparticles encapsulating the imidazoquinoline; wherein the step of processing the structures comprises extruding the solution or suspension of multilamellar structures through membranes to form lipid shell nanoparticles encapsulating the imidazoquinoline; or by drying the solution or suspension of multilamellar structures to form a film, solubilising the film, and shaking or sonicating the resulting solution to form lipid shell nanoparticles encapsulating the imidazoquinoline; or by using microfluidic mixing technologies to form lipid shell nanoparticles encapsulating the imidazoquinolines.

24-26. (canceled)

27. The method of claim 20, further comprising a step of adding a hydrogel polymer such as PEG 4000, between step (a) and step (b), or between step (b) and step (c), or between step (c) and step (d), or after step (d).

28. The method of claim 20, wherein step (a) comprises solubilising the imidazoquinoline in aqueous solution in the presence of a hydroxyacid, such as citric acid, tartaric acid, lactic acid, glycolic acid or malic acid, optionally in the presence of a host molecule such as a cyclodextrin.

29. (canceled)

30. The method of claim 20, further comprising reducing unencapsulated imidazoquinoline from the nanoparticle formulation of (b), after step (b) and before step (c), optionally by ultracentrifugation or by diafiltration with membranes which are sized to retain the nanoparticles but permit the passage of free imidazoquinoline.
